(19) United States
(12) Patent Application Publication
(10) Pub. No.: US 2024/0299344 A1
(43) Pub. Date: Sep. 12, 2024

(54) COMPOUNDS, COMPOSITIONS, AND METHODS OF TREATMENT THEREOF

(71) Applicant: LifeMine Therapeutics, Cambridge, MA (US)

(72) Inventors: Gregory L. Verdine, Cambridge, MA (US); Jared N. Cumming, Cambridge, MA (US); Susanne M. Saalau, Cambridge, MA (US); M. Paola Castaldi, Cambridge, MA (US); Yoon Choi, Cambridge, MA (US)

(21) Appl. No.: 18/549,061

(22) PCT Filed: Mar. 4, 2022
(86) PCT No.: PCT/US2022/018892

§ 371 Date: Sep. 5, 2023

Related U.S. Application Data
(60) Provisional application No. 63/156,880, filed on Mar. 4, 2021.

(51) Int. Cl. A61K 31/365; A61P 35/00

(52) U.S. Cl. CPC A61K 31/365; A61P 35/00

(57) ABSTRACT

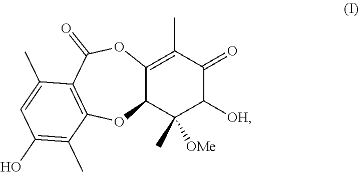

The present disclosure relates to treatment of a subject comprising administering to the subject a compound of formula (I); or a pharmaceutically acceptable salt thereof.

### FIELD OF THE PRESENT DISCLOSURE

The present disclosure relates to compounds of Formula (I) and pharmaceutically acceptable salts thereof, to pharmaceutical compositions comprising such compounds and salts, and to the uses thereof. The compounds, salts and compositions of the present disclosure may be useful for the treatment of abnormal cell growth, such as cancer, in a subject.

### BACKGROUND

Cyclin-dependent kinases (CDKs) and related serine/threonine protein kinases are important cellular enzymes that perform essential functions in regulating cell division and proliferation. CDKs 1-4, 6, 10, 11 have been reported to play a direct role in cell cycle progression, while CDKs 5 and 7-9 may play an indirect role (e.g., through activation of other CDKs, regulation of transcription or neuronal functions). The CDK catalytic units are activated by binding to regulatory subunits, known as cyclins, followed by phosphorylation. Cyclins upon expression regulate progressive phases of the cell cycle (G1, S, G2 and M) which serve as important checkpoints. CDK1/cyclin B, CDK2/cyclin A, CDK2/cyclin E, CDK4/cyclin D, CDK6/cyclin D, and likely other heterodynes are important regulators of cell cycle progression.

Overexpression of CDK2/cyclin E is associated with abnormal regulation of the cell-cycle. The CDK2/cyclin E complex plays and important role in regulation of the G1/S transition, histone biosynthesis and centrosome duplication. Progressive phosphorylation of retinoblastoma (Rb) by CDK4/6/cyclin D and CDK2/cyclin E releases the G1 transcription factors, E2Fs, and promotes S-phase entry. Activation of CDK2/cyclin A during early S-phase promotes phosphory lation of endogenous substrates that permit DNA replication and activation of E2Fs, for S-phase progression. (Asghar et al. The history and future of targeting cyclin-dependent kinases in cancer therapy, Nat. Rev. Drug. Discov. 2015: 14(2): 130-146).

There remains a need to discover CDK inhibitors having novel activity profiles, such as selective CDK2 inhibitors, which may be useful for the treatment of cancer or other proliferative diseases or conditions. In particular, CDK2 inhibitors may be useful in treating CCNE1 or CCNE2 amplified tumors.

### SUMMARY

The present disclosure includes, among other things, a method of treating a disorder in a subject, comprising administering to the subject a therapeutically effective amount of a compound of Formula (I):

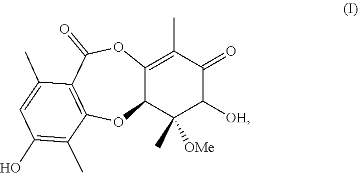 [SMILES: CO[C@@]1(C)C(O)C(=O)C(C)=C2OC(=O)c3c(C)cc(O)c(C)c3O[C@@H]21]

or a pharmaceutically acceptable salt thereof.

### Definitions

In the present disclosure, the following terms have the following meanings:

The term “about”, preceding a figure encompasses plus or minus 10%, or less, of the value of said figure. It is to be understood that the value to which the term “about” refers is itself also specifically, and preferably, disclosed.

The term “additional anticancer therapeutic agent” as used herein means any one or more therapeutic agent, other than a compound of the present disclosure, that is or can be used in the treatment of cancer. In some embodiments, such additional anticancer therapeutic agents include compounds derived from the following classes: mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, Parp inhibitors, biological response modifiers, enzyme inhibitors, antisense oligonucleotides or oligonucleotide derivatives, cytotoxics, immuno-oncology agents, and the like. In some embodiments, the additional anticancer agent is an endocrine agent, such as an aromatase inhibitor, a SERD or a SERM.

The term “administration”, or a variant thereof (e.g. “administering”), means providing the active agent or active ingredient, alone or as part of a pharmaceutically acceptable composition, to the patient in whom/which the condition, symptom, or disease is to be treated or prevented.

“Ameliorating” means a lessening or improvement of one or more symptoms upon treatment with a combination described herein, as compared to not administering the combination. “Ameliorating” also includes shortening or reduction in duration of a symptom.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous). Abnormal cell growth includes the abnormal growth of: (1) tumor cells (tumors) that show increased expression of CDK2: (2) tumors that proliferate by aberrant CDK2 activation: (3) tumors characterized by amplification or overexpression of CCNE1 and/or CCNE2; and (4) tumors that are resistant to endocrine therapy, HER2 antagonists or CDK4/6 inhibition.

The term “biological sample”, as used herein, includes, without limitation, cell cultures or extracts thereof: biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

The term “inhibitor” refers to a natural or synthetic compound that has a biological effect to inhibit or significantly reduce or down-regulate the expression of a gene and/or a protein or that has a biological effect to inhibit or significantly reduce the biological activity of a protein.

As used herein, the term “combination” preferably means a combined occurrence of the two or more therapeutic agents. In some embodiments, a combination of the present disclosure may occur either as one composition, comprising all the components in one and the same mixture (e.g. a pharmaceutical composition), or may occur as a kit of parts, wherein the different components form different parts of such a kit of parts. Administration of each compound of a combination of the present disclosure may occur either simultaneously or timely staggered, with similar or different timing of administration (i.e. similar or different numbers of administration of each component), either at the same site of administration or at different sites of administration, under similar of different dosage form.

The term “chemotherapy” refers to a type of cancer treatment that uses one or more anti-cancer drugs (chemotherapeutic agents) as part of a standardized chemotherapy regimen. Chemotherapy may be given with a curative intent or it may aim to prolong life or to reduce symptoms. Chemotherapeutic agents are for example selected from anticancer alkylating agents, anticancer antimetabolites, anticancer antibiotics, plant-derived anticancer agents, anti-cancer platinum coordination compounds and any combination thereof.

The term “hormone therapy” refers to the use of hormones in medical treatment. In one embodiment, the hormone therapy is oncologic hormone therapy.

The term “human” refers to a subject of both genders and at any stage of development (i.e. neonate, infant, juvenile, adolescent, adult).

The term “subject” and “patient” are used interchangeably and refer to a mammal, more preferably a human, who/which is awaiting the receipt of, or is receiving medical care or is/will be the object of a medical procedure. In one embodiment, the subject is diagnosed with a cancer. In one embodiment, the subject is a patient, preferably a human patient, who/which is awaiting the receipt of, or is receiving, medical care or was/is/will be the subject of a medical procedure or is monitored for the development or progression of a disease, such as a cancer. In one embodiment, the subject is a human patient who is treated and/or monitored for the development or progression of a cancer. In one embodiment, the subject is a male. In another embodiment, the subject is a female. In one embodiment, the subject is an adult. In another embodiment, the subject is a child.

The expression “pharmaceutically acceptable” refers to the ingredients of a pharmaceutical composition are compatible with each other and not deleterious to the subject to which it is administered.

The expression “pharmaceutically acceptable carrier, diluent, excipient and/or adjuvant” refers to a substance that does not produce an adverse, allergic or other untoward reaction when administered to an animal, preferably a human. It includes any and all inactive substance such as for example solvents, cosolvents, antioxidants, surfactants, stabilizing agents, emulsifying agents, buffering agents. pH modifying agents, preserving agents (or preserving agents), antibacterial and antifungal agents, isotonifiers, granulating agents or binders, lubricants, disintegrants, glidants, diluents or fillers, adsorbents, dispersing agents, suspending agents, coating agents, bulking agents, release agents, absorption delaying agents, sweetening agents, flavoring agents and the like. For human administration, preparations should meet sterility, pyrogenicity, general safety and purity standards as required by regulatory offices, such as, e.g . . . . FDA Office or EMA.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al . . . describe pharmaceutically acceptable salts in detail in J. Pharmaceutical Sciences. 1977. 66. 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄-alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “prodrug” as used herein means the pharmacologically acceptable derivatives of compounds of Formula (I), such as for example esters or amides, whose in vivo biotransformation product generates the biologically active drug. Prodrugs are generally characterized by increased bio-availability and are readily metabolized into biologically active compounds in vivo.

The term “radiation therapy” refers to a method of treatment of cancer employing various radiations such as X-ray, gamma-ray, neutron ray, electron beam, proton beam and radiation sources. It is used as part of cancer treatment to control or kill malignant cells. Radiation therapy may be curative in a number of types of cancer if they are localized to one area of the body. It may also be used as part of adjuvant therapy, to prevent tumor recurrence after surgery to remove a primary malignant tumor. The three main divisions of radiation therapy are: external beam radiation therapy (EBRT or XRT): brachytherapy or sealed source radiation therapy; and systemic radioisotope therapy (RIT) or unsealed source radiotherapy.

The terms “therapeutically effective amount” or “effective amount” or “therapeutically effective dose” refer to the amount or dose of active ingredient that is aimed at, without causing significant negative or adverse side effects to the subject, (1) delaying or preventing the onset of a cancer in the subject: (2) reducing the severity or incidence of a cancer: (3) slowing down or stopping the progression, aggravation, or deterioration of one or more symptoms of a cancer affecting the subject: (4) bringing about ameliorations of the symptoms of a cancer affecting the subject: or (5) curing a cancer affecting the subject. A therapeutically effective amount may be administered prior to the onset of a cancer for a prophylactic or preventive action. Alternatively, or additionally, a therapeutically effective amount may be administered after initiation of a cancer for a therapeutic action.

The terms “treat”, “treatment” or “treating” mean to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition.

The terms “treatment regimen”, “dosing protocol” and “dosing regimen” are used interchangeably to refer to the dose and timing of administration of each compound of the present disclosure, alone or in combination with another therapeutic agent.

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukemia's (cancers of the blood) generally do not form solid tumors (National Cancer Institute, Dictionary of Cancer Terms).

“Tumor burden” or “tumor load”, refers to the total amount of tumorous material distributed throughout the body. Tumor burden refers to the total number of cancer cells or the total size of tumor(s), throughout the body, including lymph nodes and bone marrow. Tumor burden can be determined by a variety of methods known in the art, such as, e.g., using calipers, or while in the body using imaging techniques, e.g., ultrasound, bone scan, computed tomography (CT), or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CR or MRI scans.

The term “stem cell transplant” refers to a procedure in which a patient receives healthy blood-forming cells (stem cells) to replace their own that have been destroyed by disease or by the radiation or high doses of anticancer drugs that are given as part of the procedure. The healthy stem cells may come from the blood or bone marrow of the patient, from a donor, or from the umbilical cord blood of a newborn baby. A stem cell transplant may be autologous (using a patient's own stem cells that were collected and saved before treatment), allogeneic (using stem cells donated by someone who is not an identical twin), or syngeneic (using stem cells donated by an identical twin).

### DETAILED DESCRIPTION

### Compounds

In some embodiments, the present disclosure includes a compound of Formula (I):

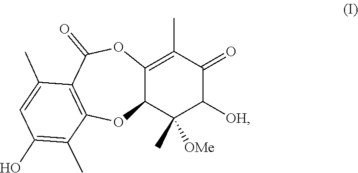 [SMILES: CO[C@@]1(C)C(O)C(=O)C(C)=C2OC(=O)c3c(C)cc(O)c(C)c3O[C@@H]21]

or a pharmaceutically acceptable salt thereof.

In some embodiments, a compound of formula (I) is

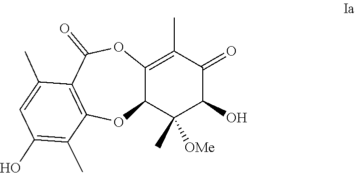 [SMILES: CO[C@@]1(C)[C@H](O)C(=O)C(C)=C2OC(=O)c3c(C)cc(O)c(C)c3O[C@@H]21]

or a pharmaceutically acceptable salt thereof.

In some embodiments, a compound of formula (I) is

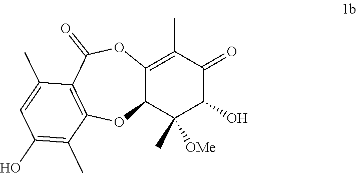

or a pharmaceutically acceptable salt thereof.

In some embodiments a CDK2 inhibitor is a compound of formula (I). In some embodiments a CDK2 inhibitor is compound 1a. In some embodiments a CDK2 inhibitor is compound 1b.

### Detection of CDK2

In some embodiments, the present disclosure provides a method to determine if a subject has an elevated level of CDK2 expression comprising: (a) detecting the level of CDK2 expression in a sample from the subject using an in vitro assay and (b) comparing the level of the CDK2 expression to a suitable reference level of CDK2 expression. In some embodiments, a subject with abnormal CDK2 expression is administered a compound or a combination of compounds effective for treatment of a patient having a high level of CDK2 expression.

In some embodiments, CDK2 expression may be determined using a suitable assay. In some embodiments, CDK2 expression in patient tumor material is determined though immunohistology, which includes immunohistochemistry (IHC) and/or immunofluorescent (IF) approaches. This includes making thin sections from a core biopsy and staining with CDK2 antibodies. Secondary antibodies are used that are either fused to an enzyme (IHC: often a peroxidase) or a fluorophore (IF) for visualization. Slides are scored by microscope and compared to healthy adjacent tissue to determine expression level.

In some embodiments CDK2 expression of a tumor cell is compared to a suitable standard. In some embodiments, a suitable standard is CDK2 expression level in a subject not diagnosed with cancer. In some embodiments, a suitable standard is CDK2 expression level of a sample from the subject from which CDK2 expression of a tumor cell is being measured. In some embodiments, a suitable standard is CDK2 expression level of a non-cancerous cellular sample adjacent to a tumor from the subject.

In some embodiments, the present disclosure includes determining a level of CDK2 expression in a tumor in a subject comprising
obtaining or having obtained a biological sample from the subject; and
performing an assay on the biological sample to determine if the tumor cells express an elevated level of CDK2 expression.

In some embodiments, the present disclosure includes determining a level of CDK2 expression in a tumor in a subject comprising
obtaining or having obtained a biological sample from the subject; and
performing an assay on the biological sample to determine if the tumor cells express an abnormal level of CDK2 expression.

### Detection of CCNE1

In some embodiments, the present disclosure provides a method to determine if a subject has an elevated level of CCNE1 expression comprising: (a) detecting the level of CCNE1 expression in a sample from the subject using an in vitro assay and (b) comparing the level of the CCNE1 expression to a suitable reference level of CCNE1 expression. In some embodiments, a subject with abnormal CCNE1 expression is administered a compound or a combination of compounds effective for treatment of a patient having a high level of CCNE1 expression.

In some embodiments, CCNE1 expression may be determined using a suitable assay. In some embodiments, CCNE1 expression in patient tumor material is determined though immunohistology, which includes immunohistochemistry (IHC) and/or immunofluorescent (IF) approaches. This includes making thin sections from a core biopsy and staining with Cyclin E1 antibodies. Secondary antibodies are used that are either fused to an enzyme (IHC: often a peroxidase) or a fluorophore (IF) for visualization. Slides are scored by microscope and compared to healthy adjacent tissue to determine expression level.

In some embodiments CCNE1 expression of a tumor cell is compared to a suitable standard. In some embodiments, a suitable standard is CCNE1 expression level in a subject not diagnosed with cancer. In some embodiments, a suitable standard is CCNE1 expression level of a sample from the subject from which CCNE1 expression of a tumor cell is being measured. In some embodiments, a suitable standard is CCNE1 expression level of a non-cancerous cellular sample adjacent to a tumor from the subject.

In some embodiments, the present disclosure includes determining a level of CCNE1 expression in a tumor in a subject comprising
obtaining or having obtained a biological sample from the subject; and
performing an assay on the biological sample to determine if the tumor cells express an elevated level of CCNE1 expression.

In some embodiments, the present disclosure includes determining a level of CCNE1 expression in a tumor in a subject comprising
obtaining or having obtained a biological sample from the subject; and
performing an assay on the biological sample to determine if the tumor cells express an abnormal level of CCNE1 expression.

### Detection of CCNE2

In some embodiments, the present disclosure provides a method to determine if a subject has an elevated level of CCNE2 expression comprising: (a) detecting the level of CCNE2 expression in a sample from the subject using an in vitro assay and (b) comparing the level of the CCNE2 expression to a suitable reference level of CCNE2 expression. In some embodiments, a subject with abnormal CCNE2 expression is administered a compound or a combination of compounds effective for treatment of a patient having a high level of CCNE2 expression.

In some embodiments, CCNE2 expression may be determined using a suitable assay. In some embodiments, CCNE2 expression in patient tumor material is determined though immunohistology, which includes immunohistochemistry (IHC) and/or immunofluorescent (IF) approaches. This includes making thin sections from a core biopsy and staining with Cyclin E2 antibodies. Secondary antibodies are used that are either fused to an enzyme (IHC: often a peroxidase) or a fluorophore (IF) for visualization. Slides are scored by microscope and compared to healthy adjacent tissue to determine expression level.

In some embodiments CCNE2 expression of a tumor cell is compared to a suitable standard. In some embodiments, a suitable standard is CCNE2 expression level in a subject not diagnosed with cancer. In some embodiments, a suitable standard is CCNE2 expression level of a sample from the subject from which CCNE2 expression of a tumor cell is being measured. In some embodiments, a suitable standard is CCNE2 expression level of a non-cancerous cellular sample adjacent to a tumor from the subject.

In some embodiments, the present disclosure includes determining a level of CCNE2 expression in a tumor in a subject comprising
obtaining or having obtained a biological sample from the subject; and
performing an assay on the biological sample to determine if the tumor cells express an elevated level of CCNE2 expression.

In some embodiments, the present disclosure includes determining a level of CCNE2 expression in a tumor in a subject comprising
obtaining or having obtained a biological sample from the subject; and
performing an assay on the biological sample to determine if the tumor cells express an abnormal level of CCNE2 expression.

The terms “control”, “standard” or “reference” are used interchangeably and refer to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome: normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present disclosure. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e . . . treatment naïve), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard: determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the disclosure are not limited to use of a specific cut-point in comparing the level of expression product in the test sample to the control.

### Uses

### Inhibition

In some embodiments, the compounds of the present disclosure are selective inhibitors of CDK2, i.e., they have a lower inhibitory constant (e.g., Kᵢ or IC₅₀) for CDK2 relative to other enzymatic targets. In some embodiments, the compounds of the present disclosure are selective inhibitors of CDK2/cyclin E1, i.e., they have a lower inhibitory constant (e.g., Kᵢ or IC₅₀) for CDK2/cyclin E1 relative to other enzymatic targets. In some embodiments, the compounds of the present disclosure are selective inhibitors of CDK2/cyclin E2, i.e., they have a lower inhibitory constant (e.g., Kᵢ or IC₅₀) for CDK2/cyclin E2 relative to other enzymatic targets.

### Methods

The present disclosure further provides therapeutic methods and uses comprising administering the compounds of the present disclosure, or pharmaceutically acceptable salts thereof, alone or in combination with other therapeutic agents or palliative agents.

In one aspect, the present disclosure provides a method for the treatment of abnormal cell growth in a subject in need thereof, comprising administering to the subject a therapeutically effective amount of a compound of the present disclosure, or a pharmaceutically acceptable salt thereof.

In another aspect, the present disclosure provides a method for the treatment of abnormal cell growth in a subject in need thereof, comprising administering to the subject an amount of a compound of the present disclosure, or a pharmaceutically acceptable salt thereof, in combination with an amount of an additional therapeutic agent (e.g., an anticancer therapeutic agent), which amounts are together effective in treating said abnormal cell growth.

In another aspect, the present disclosure provides a compound of the present disclosure, or a pharmaceutically acceptable salt thereof, for use in the treatment of abnormal cell growth in a subject.

In a further aspect, the present disclosure provides the use of a compound of the present disclosure, or a pharmaceutically acceptable salt thereof, for the treatment of abnormal cell growth in a subject.

In another aspect, the present disclosure provides a pharmaceutical composition for use in the treatment of abnormal cell growth in a subject in need thereof, which pharmaceutical composition comprises a compound of the present disclosure, or a pharmaceutically acceptable salt thereof, and a pharmaceutically acceptable carrier or excipient.

In another aspect, the present disclosure provides a compound of the present disclosure, or a pharmaceutically acceptable salt thereof, for use as a medicament, in particular a medicament for the treatment of abnormal cell growth.

In yet another aspect, the present disclosure provides the use of a compound of the present disclosure, or a pharmaceutically acceptable salt thereof, for the manufacture of a medicament for the treatment of abnormal cell growth in a subject.

In frequent embodiments of the methods provided herein, the abnormal cell growth is cancer. Compounds of the present disclosure may be administered as single agents or may be administered in combination with other anti-cancer therapeutic agents, in particular standard of care agents appropriate for the particular cancer.

In some embodiments, the methods provided result in one or more of the following effects; (1) inhibiting cancer cell proliferation: (2) inhibiting cancer cell invasiveness: (3) inducing apoptosis of cancer cells: (4) inhibiting cancer cell metastasis: or (5) inhibiting angiogenesis.

In another aspect, the present disclosure provides a method for the treatment of a disorder mediated by CDK2 in a subject, comprising administering to the subject a compound of the present disclosure, or a pharmaceutically acceptable salt thereof, in an amount that is effective for treating said disorder, in particular cancer.

In another aspect, the present disclosure provides a method of inhibiting cancer cell proliferation in a subject, comprising administering to the subject a compound of the present disclosure, or a pharmaceutically acceptable salt thereof, in an amount effective to inhibit cell proliferation.

In another aspect, the present disclosure provides a method of inhibiting cancer cell invasiveness in a subject, comprising administering to the subject a compound of the present disclosure, or a pharmaceutically acceptable salt thereof, in an amount effective to inhibit cell invasiveness.

In another aspect, the present disclosure provides a method of inducing apoptosis in cancer cells in a subject, comprising administering to the subject a compound of the present disclosure, or a pharmaceutically acceptable salt thereof, in an amount effective to induce apoptosis.

In another aspect, the present disclosure provides a method of inhibiting cancer cell metastasis in a subject, comprising administering to the subject a compound of the present disclosure, or a pharmaceutically acceptable salt thereof, in an amount effective to inhibit cell metastasis.

In another aspect, the present disclosure provides a method of inhibiting angiogenesis in a subject, comprising administering to the subject a compound of the present disclosure, or a pharmaceutically acceptable salt thereof, in an amount effective to inhibit angiogenesis.

In some embodiments, a compound of the present disclosure is administered as first line therapy. In other embodiments, a compound of the present disclosure is administered as second (or later) line therapy. In some embodiments, a compound of the present disclosure is administered as second (or later) line therapy following treatment with an endocrine therapeutic agent and/or a CDK4/CDK6 inhibitor. In some embodiments, a compound of the present disclosure is administered as second (or later) line therapy following treatment with an endocrine therapeutic agent, e.g., an aromatase inhibitor, a SERM or a SERD. In some embodiments, a compound of the present disclosure is administered as second (or later) line therapy following treatment with a CDK4/CDK6 inhibitor. In some embodiments, a compound of the present disclosure is administered as second (or later) line therapy following treatment with one or more chemotherapy regimens, e.g., including taxanes or platinum agents. In some embodiments, a compound of the present disclosure is administered as second (or later) line therapy following treatment with HER2 targeted agents, e.g., trastuzumab.

For the purposes of this present disclosure, beneficial or desired clinical results include, but are not limited to, one or more of the following: reducing the proliferation of (or destroying) neoplastic or cancerous cell: inhibiting metastasis or neoplastic cells: shrinking or decreasing the size of a tumor: remission of the cancer: decreasing symptoms resulting from the cancer: increasing the quality of life of those suffering from the cancer: decreasing the dose of other medications required to treat the cancer: delaying the progression of the cancer: curing the cancer; overcoming one or more resistance mechanisms of the cancer; and/or prolonging survival of patients the cancer. Positive therapeutic effects in cancer can be measured in a number of ways (see, for example, W. A. Weber, Assessing tumor response to therapy, J. Nucl. Med. 50 Suppl. 1:1S-10S (2009). For example, with respect to tumor growth inhibition (T/C), according to the National Cancer Institute (NCI) standards, a T/C less than or equal to 42% is the minimum level of anti-tumor activity. A T/C<10% is considered a high anti-tumor activity level, with T/C (%)=median tumor volume of the treated/median tumor volume of the control x 100.

In some embodiments, the treatment achieved by a compound of the present disclosure is defined by reference to any of the following: partial response (PR), complete response (CR), overall response (OR), progression free survival (PFS), disease free survival (DFS) and overall survival (OS). PFS, also referred to as “Time to Tumor Progression” indicates the length of time during and after treatment that the cancer does not grow and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naïve or untreated subjects or patients. In some embodiments, response to a combination of the present disclosure is any of PR, CR. PFS, DFS, OR or OS that is assessed using Response Evaluation Criteria in Solid Tumors (RECIST) 1.1 response criteria.

The treatment regimen for a compound of the present disclosure that is effective to treat a cancer patient may vary according to factors such as the disease state, age, and weight of the patient, and the ability of the therapy to elicit an anti-cancer response in the subject. While an embodiment of any of the aspects of the present disclosure may not be effective in achieving a positive therapeutic effect in every subject, it should do so in a statistically significant number of subjects as determined by any statistical test known in the art such as the Student's t-test, the chi2-test the U-test according to Mann and Whitney, the Kruskal-Wallis test (H-test), Jonckheere-Terpstrat-testy and the Wilcon on-test.

In some embodiments, the present disclosure includes a method of treating cancer characterized by elevated CDK2 expression in a subject in need thereof, comprising administering to the subject a therapeutically effective amount of a compound of the present disclosure. In some embodiments, the present disclosure includes a method of treating cancer characterized by elevated CDK2 expression in tumor cells in a subject in need thereof, comprising administering to the subject a therapeutically effective amount of a compound of the present disclosure.

In some embodiments, the present disclosure includes a method of treating cancer in a subject in need thereof, comprising administering to the subject a therapeutically effective amount of a compound of the present disclosure, wherein the subject has previously been identified as having elevated CDK2 expression in tumor cells of the subject. In some embodiments, a subject has previously been identified as having elevated CDK2 expression in the tumor microenvironment as compared to a normal adjacent tissue. In some embodiments, a subject has previously been identified as having elevated CDK2 expression in the tumor microenvironment as compared to a reference. In some embodiments, a subject has previously been identified as having elevated CDK2 expression in the tumor microenvironment as compared to a standard.

In some embodiments, the present disclosure includes a method of treating cancer in a subject in need thereof, comprising:
selecting a subject with cancer having a diagnosis of an elevated CDK2 expression in tumor cells of the subject; and
treating the patient with a compound of the present disclosure.

In some embodiments, a subject with cancer has a diagnosis of an elevated CDK2 expression in tumor cells of the subject as compared to a reference. In some embodiments, a subject with cancer has a diagnosis of an elevated CDK2 expression in tumor cells of the subject as compared to a standard. In some embodiments, a subject with cancer has a diagnosis of an elevated CDK2 expression in tumor cells of the subject as compared to normal adjacent cells.

In some embodiments, the present disclosure includes a method of treating cancer in a subject in need thereof, comprising:
selecting a subject with cancer having a diagnosis of an elevated CDK2 expression in tumor cells of the subject; and
treating the patient with a compound of the present disclosure.

In some embodiments, a subject with cancer has a diagnosis of an elevated CDK2 expression in tumor cells of the subject as compared to a reference. In some embodiments, a subject with cancer has a diagnosis of an elevated CDK2 expression in tumor cells of the subject as compared to a standard. In some embodiments, a subject with cancer has a diagnosis of an elevated CDK2 expression in tumor cells of the subject as compared to normal adjacent cells.

In some embodiments, the present disclosure includes a method of treating cancer characterized by elevated cyclin E expression in a subject in need thereof, comprising administering to the subject a therapeutically effective amount of a compound of the present disclosure. In some embodiments, the present disclosure includes a method of treating cancer characterized by elevated cyclin E expression in tumor cells in a subject in need thereof, comprising administering to the subject a therapeutically effective amount of a compound of the present disclosure.

In some embodiments, the present disclosure includes a method of treating cancer in a subject in need thereof, comprising administering to the subject a therapeutically effective amount of a compound of the present disclosure, wherein the subject has previously been identified as having elevated cyclin E expression in tumor cells of the subject. In some embodiments, a subject has previously been identified as having elevated cyclin E expression in the tumor microenvironment as compared to a normal adjacent tissue. In some embodiments, a subject has previously been identified as having elevated cyclin E expression in the tumor microenvironment as compared to a reference. In some embodiments, a subject has previously been identified as having elevated cyclin E expression in the tumor microenvironment as compared to a standard.

In some embodiments, the present disclosure includes a method of treating cancer in a subject in need thereof, comprising:
selecting a subject with cancer having a diagnosis of an elevated cyclin E expression in tumor cells of the subject; and
treating the patient with a compound of the present disclosure.

In some embodiments, a subject with cancer has a diagnosis of an elevated cyclin E expression in tumor cells of the subject as compared to a reference. In some embodiments, a subject with cancer has a diagnosis of an elevated cyclin E expression in tumor cells of the subject as compared to a standard. In some embodiments, a subject with cancer has a diagnosis of an elevated cyclin E expression in tumor cells of the subject as compared to normal adjacent cells.

In some embodiments, the present disclosure includes a method of treating cancer in a subject in need thereof, comprising:
selecting a subject with cancer having a diagnosis of an elevated cyclin E expression in tumor cells of the subject; and
treating the patient with a compound of the present disclosure.

In some embodiments, a subject with cancer has a diagnosis of an elevated cyclin E expression in tumor cells of the subject as compared to a reference. In some embodiments, a subject with cancer has a diagnosis of an elevated cyclin E expression in tumor cells of the subject as compared to a standard. In some embodiments, a subject with cancer has a diagnosis of an elevated cyclin E expression in tumor cells of the subject as compared to normal adjacent cells.

### Diseases and Disorders

In some embodiments, the present disclosure includes the methods of treating proliferative disorders, including cancers. In some embodiments, the present disclosure includes a compound for use in the treatment and/or prevention of proliferative disorders, including cancers. Thus, in one embodiment, the present disclosure provides use of a compound for the manufacture of a medicament for treating and/or preventing cancer. The present disclosure also provides a method of treatment of cancer, which comprises administering to a mammal species in need thereof a therapeutically effective amount of a compound.

The present disclosure also provides for a method for delaying in patient the onset of cancer comprising the administration of a pharmaceutically effective amount of a compound of the disclosure to a patient in need thereof.

Various cancers are known in the art. Cancers that can be treated using methods of the disclosure include solid cancers and non-solid cancers, especially benign and malignant solid tumors and benign and malignant non-solid tumors. Cancer may be metastatic or non-metastatic. The cancer may be familial or sporadic.

In some embodiments, cancer is a solid cancer. As used herein, the term “solid cancer” encompasses any cancer (also referred to as malignancy) that forms a discrete tumor mass, as opposed to cancers (or malignancies) that diffusely infiltrate a tissue without forming a mass.

In frequent embodiments of the methods provided herein, abnormal cell growth is cancer. In some such embodiments, cancer is selected from breast cancer, ovarian cancer, bladder cancer, uterine cancer, prostate cancer, lung cancer (including non-small cell lung cancer (NSCLC), or small cell lung cancer (SCLC)), esophageal cancer, head and neck cancer, colorectal cancer, kidney cancer (including renal cell carcinoma (RCC)), liver cancer (including hepatocellular carcinoma (HCC)), pancreatic cancer, stomach (i.e., gastric) cancer or thyroid cancer. In further embodiments of the methods provided herein, the cancer is breast cancer, ovarian cancer, bladder cancer, uterine cancer, prostate cancer, lung cancer, esophageal cancer, liver cancer, pancreatic cancer or stomach cancer.

In some embodiments, a cancer is selected from the group consisting of breast cancer, ovarian cancer, gastric cancer, endometrial cancer, bladder cancer, prostate cancer, liver cancer, glioblastoma cancer, colon cancer, neuroblastoma, oral cancer, prostate cancer, and B cell lymphoma. In some embodiments, cancer is breast cancer, ovarian cancer, gastric cancer, endometrial cancer, bladder cancer, prostate cancer, liver cancer, glioblastoma cancer, colon cancer. In some embodiments, breast cancer, oral cancer and prostate cancer. In some embodiments, cancer is selected from the group consisting of neuroblastoma, colon cancer and B cell lymphoma. In some embodiments, cancer is selected from the group consisting of lung cancer, breast cancer and colorectal cancer. In some embodiments, cancer is breast cancer. In some embodiments, cancer is breast cancer, wherein a subject has acquired resistance to a CDK4/6 inhibitor or Tamoxifen.

In some embodiments, cancer is selected from breast cancer, ovarian cancer, gastric cancer, endometrial cancer, esophageal cancer, bladder cancer, prostate cancer, liver cancer, glioblastoma, colon cancer, neuroblastoma, oral cancer, prostate cancer, skin cancer, adenocarcinoma, and lymphoma. In some embodiments, lung cancer is selected from the group consisting of NSCLC, and SCLC. In some embodiments, kidney cancer is RCC In embodiments, liver cancer is HCC. In some embodiments, cancer is adenocarcinoma. In some embodiments, skin cancer is melanoma or squamous cell carcinoma.

In other embodiments, the cancer is breast cancer, including, e.g., ER-positive/HR-positive, HER2-negative breast cancer: ER-positive/HR-positive, HER2-positive breast cancer: triple negative breast cancer (TNBC): or inflammatory breast cancer. In some embodiments, the breast cancer is endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments of each of the foregoing, the breast cancer is characterized by amplification or overexpression of CCNE1 and/or CCNE2.

In some embodiments of the methods provided herein, abnormal cell growth is cancer characterized by amplification or overexpression of CCNE1 and/or CCNE2. In some embodiments of the methods provided herein, the subject is identified as having a cancer characterized by amplification or overexpression of CCNE1 and/or CCNE2.

In some embodiments, the cancer is selected from the group consisting of breast cancer and ovarian cancer. In some such embodiments, the cancer is breast cancer or ovarian cancer characterized by amplification or overexpression of CCNE1 and/or CCNE2. In some such embodiments, the cancer is (a) breast cancer or ovarian cancer: (b) characterized by amplification or overexpression of cyclin E1 (CCNE1) or cyclin E2 (CCNE2); or (c) both (a) and (b). In some embodiments, the cancer is ovarian cancer.

### Combinations

In other embodiments, a compound of the present disclosure may be administered in combination with a standard of care agent. In some embodiments, a compound of the present disclosure may be administered in combination with therapeutic agent or treatment. . . . In some embodiments, a compound of the present disclosure is administered in combination with an endocrine therapeutic agent and/or a CDK4/CDK6 inhibitor. In some embodiments, a compound of the present disclosure is administered in combination with an endocrine therapeutic agent, e.g., an aromatase inhibitor, a SERM or a SERD. In some embodiments, a compound of the present disclosure is administered in combination with a CDK4/CDK6 inhibitor. In some embodiments, a compound of the present disclosure is administered in combination with one or more chemotherapy regimens, e.g., including taxanes or platinum agents. In some embodiments, a compound of the present disclosure is administered in combination with HER2 targeted agents, e.g., trastuzumab.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably modulate CDK2, or a mutant thereof, in a biological sample or in a patient. In certain embodiments, the amount of compound in compositions of this disclosure is such that is effective to measurably modulate CDK2, or a mutant thereof, in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents.

The term “parenteral” as used herein includes subcutaneous, intravenous, intramuscular, intra-articular, intra-synovial, intrasternal, intrathecal, intrahepatic, intralesional and intracranial injection or infusion techniques.

### Exemplification

### Example 1

Compound 1a ((5aS,6S,7S)-3,7-dihydroxy-6-methoxy-1,4,6,9-tetramethyl-6,7-dihydro-11H-dibenzo[b,e][1,4]dioxepine-8,11(5aH)-dione) and Compound 1b ((5aS,6S,7R)-3,7-dihydroxy-6-methoxy-1,4,6,9-tetramethyl-6,7-dihydro-11H-dibenzo[b,e][1,4]dioxepine-8,11(5aH)-dione can be obtained as described by Z. Shang, et al. (Organic Letters 2016, 18, 4340-4343, doi: 10.1021/acs.orglett.6b02099).

### Isolation

For a 45 mL scale fermentation, the culture broth was transferred to a conical tube and subjected to centrifugation (20 min). The supernatant was then decanted into ethyl acetate (200 mL). The mixture was shaken for 4 h and allowed to stand overnight, and the organic layer was then separated dried under reduced pressure.

For a 450 mL scale fermentation, the culture broth was transferred to a bucket centrifuge and subjected to centrifugation. The supernatant was then decanted into ethyl acetate (500 mL) and stirred overnight. The organic layer was separated and dried under reduced pressure to give a crude residue.

Each crude residue was resuspended in acetonitrile, centrifuged, and filtered. Several of these filtered samples from both 45 mL scale and 450 mL scale fermentations were combined and re-concentrated under reduced pressure to give a single crude mixture of Compound 1a and Compound 1b (400 mg).

This crude mixture was subjected to reverse-phase high performance liquid chromatography (HPLC) (Waters Sunfire C18 column, 10×150 mm, C18, 5 μm, gradient from 95:5 to 0:100 water:acetonitrile). Fractions containing Compound 1a and Compound 1b were separately concentrated under reduced pressure to give samples of Compound 1a and Compound 1b. These samples were then independently subjected to additional chromatography as follows: (1) reverse-phase HPLC (Phenomenex Kinetex F5 core-shell column, 21.2×250 mm, 5 μm, gradient from 95:5 to 0:100 water:acetonitrile with 0.1% formic acid), (2) concentration, (3) reverse-phase HPLC (Waters XSelect CSH C18 column, 10×250 mm, 5 μm, fluoro-phenyl, gradient from 95:5 to 0:100 water: acetonitrile with 0.1% formic acid), (4) concentration to afford product.

Compound 1a (C₁₈H₂₀O₇, 0.5 mg): HRESIMS (m z) 349.1279 [M+H]⁺ (calculated for C₁₈H₂₁O₇, 349.1282): ¹H NMR (600 MHZ, tetrahydrofuran-d8) δ (ppm)=6.53-6.51 (m, 1H), 5.06 -5.04 (m, 1H), 4.25-4.23 (m, 1H), 3.44 (s, 3H), 2.44 (s, 3H), 2.18 (s, 3H), 1.86 (d, J=2.0 Hz, 3H), 1.29 (s, 2H), 1.15 (s, 3H): ¹³C NMR (151 MHZ, tetrahydrofuran-d8) δ (ppm)=197.9, 162.5, 162.2, 161.7, 161.3, 143.8, 117.3, 115.3, 115.2, 112.5, 84.5, 83.0, 77.7, 51.4, 30.7, 22.5, 12.4, 8.6.

Compound 1b (C₁₈H₂₀O₇, 0.25 mg): HRESIMS (m z) 349.1280 [M+H]⁺ (calculated for C₁₈H₂₁O₇, 349.1282): ¹H NMR (600 MHZ, tetrahydrofuran-d8) δ (ppm)=9.16 (br s, 1H), 6.43 (s, 1H), 4.90 (s, 1H), 4.37 (s, 1H), 3.33 (s, 3H), 2.36 (s, 3H), 2.08 (s, 3H), 1.66 (s, 3H): ¹³C NMR (151 MHz, tetrahydrofuran-d8) δ (ppm)=197.3, 162.9, 160.3, 157.1, 156.9, 141.1, 117.9, 113.5, 112.3, 109.9, 81.0, 78.8, 77.2, 52.3, 22.8, 16.3, 9.0, 7.9.

### Example 2

Protein kinases CDK2/cyclin A1, CDK2/cyclin A2, CDK2/cyclin E1, CDK2/cyclin E2 and CDK2/cyclin O were screened via radiometric HotSpot™ assay at Reaction Biology Corporation, Malvern, PA. The buffer for the enzyme reactions was: 20 mM Hepes (pH 7.5), 10 mM MgCl₂, 1 mM EGTA, 0.01% Brij35, 0.02 mg/mL BSA, 0.1 mM Na₃VO₄, 2 mM DTT, 1% DMSO. The substrate was prepared fresh before the reaction. The enzyme was added to the substrate and gently mixed. Compounds were dissolved in DMSO and diluted in 100% DMSO in a half log dilution series. These were added to the substrate/enzyme mixture. The reaction was initiated with addition of ³³P ATP for a final concentration of 10 μM in the well. The reactions were incubated at room temperature for 2 hours. Kinase activity was detected by P81 filter binding readout method. Data was normalized to no inhibitor (DMSO vehicle) for the high controls and no enzyme (+DMSO vehicle) for the low controls.

| Enzyme (Concentration) | Substrate (Concentration) |
|---|---|
| CDK2/cyclin A2 (0.15 nM) | RB Protein (3 μM) |
| CDK2/cyclin A1 (2.0 nM) | RB Protein (3 μM) |
| CDK2/cyclin E1 (1.5 nM) | RB Protein (3 μM) |
| CDK2/cyclin E2 (2.0 nM) | RB Protein (3 μM) |
| CDK2/cyclin O (5.0 nM) | Histone H1 (20 μM) |

Reference: Anastassiadis, T., et al. “Comprehensive assay of kinase catalytic activity reveals features of kinase inhibitor selectivity” Nature Biotechnology 2011, 29, 1039-1045, doi: 10.1038/nbt.2017.

Compound 1a and Compound 1b were tested in the above described assay and demonstrated the following kinase inhibitory activity:

Example 3

| | Compound 1a | Compound 1b |
|---|---|---|
| Kinase | IC₅₀ (μM) | IC₅₀ (μM) |
| CDK2/cyclin A2 | 2.6 | 6.2 |
| CDK2/cyclin A1 | 1.1 | 2.4 |
| CDK2/cyclin E1 | 0.56 | 1.4 |
| CDK2/cyclin E2 | 0.32 | 0.76 |
| CDK2/cyclin O | 1.8 | 3.5 |

Cells from an ovarian cancer cell line, OVCAR3, were seeded at 3000 cells/well in 96-well plates in Roswell Park Memorial Institute (RPMI) media supplemented with 20% fetal bovine serum (FBS)+10 ug/mL insulin for 24 hours at 37° C. and 5% CO₂. The compounds were serially diluted from a 10 mM DMSO stock and tested at top dose of 33 μM with 3-fold dilution in a 9-point dose curve. Cells were incubated for 5 days at 37° C. and 5% CO₂. To determine the relative growth upon treatment, CellTiter-Glo® 2.0 Viability Assay (Promega™, Madison, WI) was performed per manufacturer's specifications and luminescence was read on a ClarioSTARI plate reader. Mean GIso values were determined using a four-parameter fit determined by GraphPad Prism software.

Compound 1a and Compound 1b were tested in the above described assay and demonstrated the following kinase inhibitory activity:

Example 4

| | CellTiter-Glo GI₅₀ (μM) |
|---|---|
| Compound la | 1.8 |
| Compound 1b | 3.3 |

HEK293 cells were co-transfected with CDK2-NanoLuc® Fusion Vector (CDK2: NV2781) and CCNE1 Expression Vector (CCNE1: NV2781) (Promega™, Madison, WI). Cells were seeded at 20,000 cells/well in 96-well plates in Dulbecco's Modified Eagle Medium (DMEM) supplemented with 10% FBS. The compounds were serially diluted from a 10 mM DMSO stock and tested at a top concentration of 33 μM with 3-fold dilution in a 10-point dose curve. Cells were incubated for 2 hours at 37° C. and 5% CO₂ and performed per manufacturer's specifications (Promega™, Madison, WI). Tracer #10 (K-10; N2641) was added in appropriate wells. NanoBRET NanoGlo Substrate and Extracellular NanoLuc Inhibitor were added, and donor (450 nm) and acceptor (610 nm) emission were measured within 10 minutes on a ClarioStar 2 plate reader. Mean corrected milliBRET units (mBU) was calculated as following:

### NanoBRETT Ratio Equation:

$$BRET Ratio = \frac{{Acceptor}_{sample}}{{Donor}_{sample}} \times 1 , 000$$

### NanoBRETT& Ratio Equation, Including Optional Background Correction:

$$BRET Ratio = [ ( \frac{{Acceptor}_{sample}}{{Donor}_{sample}} ) - ( \frac{{Acceptor}_{\text{?}}}{{Donor}_{\text{?}}} ) ] \times 1 , 000$$

$$\text{?} \text{indicates text missing or illegible when filed}$$

Compound 1a and Compound 1b were tested in the above described assay and demonstrated the following kinase inhibitory activity:

| | NanoBRET IC₅₀ (μM) |
|---|---|
| Compound 1a | 0.20 |
| Compound 1b | 0.74 |

We claim:

1. A method of treating a disorder characterized by amplification or overexpression of cyclin E, amplification or overexpression of CDK2, amplification or overexpression of cMYC, or a KRAS mutation in a subject, comprising administering to the subject a therapeutically effective amount of a compound of Formula (I): 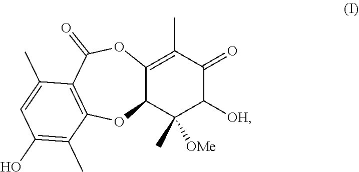
or a pharmaceutically acceptable salt thereof.

2. The method of claim 1, wherein the disorder is characterized by amplification or overexpression of cyclin E.

3. The method of any of claims 1-2, wherein the cyclin E is CCNE1.

4. The method of any of claims 1-2, wherein the cyclin E is CCNE2.

5. The method of claim 1, wherein the disorder is characterized amplification or overexpression of CDK2.

6. The method of claim 1, wherein the disorder is characterized by amplification or overexpression of cMYC.

7. The method of claim 1, wherein the disorder is characterized by a KRAS mutation.

8. The method of any of claims 1-7, wherein the disorder is cancer.

9. The method of claim 8, wherein the cancer is selected from breast cancer, ovarian cancer, gastric cancer, endometrial cancer, esophageal cancer, bladder cancer, prostate cancer, liver cancer, glioblastoma, colon cancer, neuroblastoma, oral cancer, prostate cancer, skin cancer, adenocarcinoma, and lymphoma.

10. The method of claim 9, wherein the lung cancer is selected from the group consisting of NSCLC, and SCLC.

11. The method of claim 9, wherein the kidney cancer is RCC.

12. The method of claim 9, wherein the liver cancer is HCC.

13. The method of claim 9, wherein the breast cancer is selected from the group consisting of ER-positive/HR-positive, HER2-negative breast cancer; ER-positive/HR-positive, HER2-positive breast cancer; triple negative breast cancer (TNBC); and inflammatory breast cancer.

14. The method of claim 9, wherein the cancer is glioblastoma.

15. The method of claim 9, wherein the lymphoma is B-cell lymphoma.

16. The method of claim 9, wherein the skin cancer is melanoma or squamous cell carcinoma.

17. The method of claim 9, wherein the cancer is adenocarcinoma.

18. A method of treating a disorder selected from the group consisting of breast cancer, ovarian cancer, gastric cancer, endometrial cancer, bladder cancer, prostate cancer, liver cancer, glioblastoma cancer, colon cancer, neuroblastoma, oral cancer, prostate cancer, and B cell lymphoma, in a subject, comprising administering to the subject a therapeutically effective amount of a compound of Formula (I): 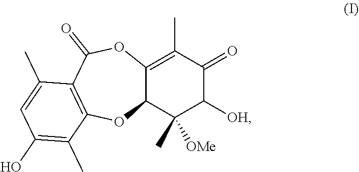
or a pharmaceutically acceptable salt thereof.

19. A method of treating cancer in a subject in need thereof, comprising
selecting a subject with cancer having a diagnosis of an elevated cyclin E expression in tumor cells of the subject; and
treating the subject with a compound of Formula (I): 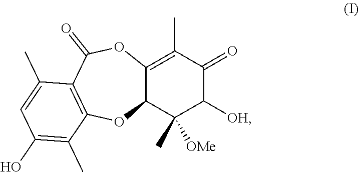
or a pharmaceutically acceptable salt thereof.

20. The method of claim 19, wherein cyclin E is CCNE2.

21. The method of claim 19, wherein cyclin E is CCNE1.

22. The method of any of claims 19-21, wherein the subject has acquired resistance to a CDK4/6 inhibitor or Tamoxifen.

23. The method of any of claims 19-22, wherein the cancer is selected from the group consisting of breast cancer, ovarian cancer, gastric cancer, endometrial cancer, bladder cancer, prostate cancer, liver cancer, glioblastoma, colon cancer.

24. The method of claim 23, wherein the cancer is breast cancer.

25. A method for treating a subject, comprising:
determining a level of cyclin E expression in tumor cells of the subject; and
administering a compound of Formula (I): 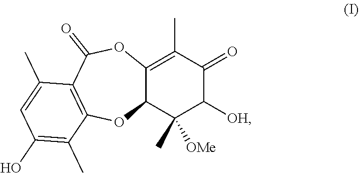
or a pharmaceutically acceptable salt thereof.

26. The method of claim 25, wherein determining the level of cyclin E expression comprises
obtaining or having obtained a biological sample from the patient; and
performing or having performed an assay on the biological sample to determine if the tumor cells express an elevated level of cyclin E expression.

27. A method of treating cancer in a subject in need thereof, comprising
selecting a subject with cancer having a diagnosis of an elevated CDK2 expression in tumor cells of the subject; and
treating the patient with a compound of Formula (I): 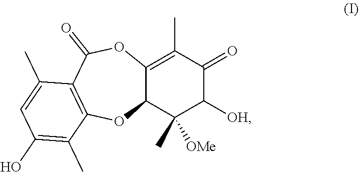
or a pharmaceutically acceptable salt thereof.

28. The method of claim 27, wherein the cancer is selected from the group consisting of breast cancer, oral cancer and prostate cancer.

29. A method for treating a subject, comprising:
determining a level of CDK2 expression in tumor cells of the subject; and
administering a compound of Formula (I): 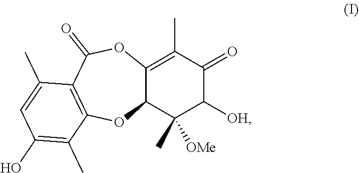
or a pharmaceutically acceptable salt thereof.

30. The method of claim 29, wherein determining said level of CDK2 expression comprises
obtaining or having obtained a biological sample from the patient; and
performing or having performed an assay on the biological sample to determine if the tumor cells express an elevated level of CDK2 expression.

31. A method of treating cancer in a subject in need thereof, comprising
selecting a subject with cancer having a diagnosis of an elevated cMYC expression in tumor cells of the subject; and
treating the patient with a compound of Formula (I): 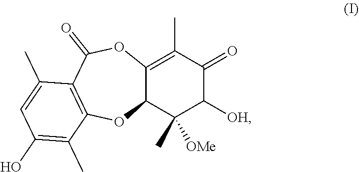
or a pharmaceutically acceptable salt thereof.

32. The method of claim 31, wherein the cancer is selected from the group consisting of neuroblastoma, colon cancer, and B cell lymphoma.

33. A method for treating a subject, comprising:
determining a level of cMYC expression in tumor cells of the subject; and
administering a compound of Formula (I): 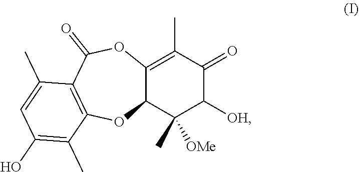
or a pharmaceutically acceptable salt thereof.

34. The method of claim 33, wherein determining said level of cMYC expression comprises
obtaining or having obtained a biological sample from the patient; and
performing or having performed an assay on the biological sample to determine if the tumor cells express an elevated level of cMYC expression.

35. A method of treating cancer in a subject in need thereof, comprising
selecting a subject with cancer having a diagnosis of presence of a KRAS mutation in tumor cells of the subject; and
treating the patient with a compound of Formula (I): 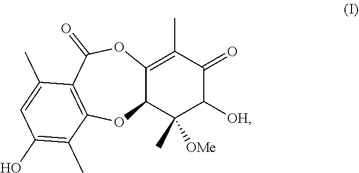
or a pharmaceutically acceptable salt thereof.

36. The method of claim 35, wherein the cancer is selected from the group consisting of lung cancer, breast cancer and colorectal cancer.

37. The method of claim 36, wherein the lung cancer is non-small cell lung cancer.

38. A method for treating a subject, comprising:
identifying a KRAS mutation in tumor in tumor cells of the subject; and
administering a compound of Formula (I): 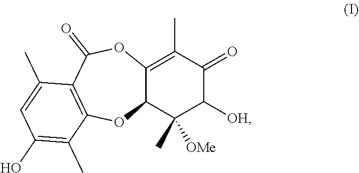
or a pharmaceutically acceptable salt thereof.

39. The method of claim 38, wherein identifying a KRAS mutation in tumor comprises
obtaining or having obtained a biological sample from the patient; and
performing or having performed an assay on the biological sample to determine if the tumor cells express mutant KRAS.

40. The method of any of claims 1-39, wherein the compound is 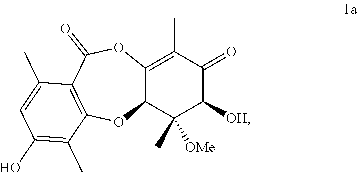
or a pharmaceutically acceptable salt thereof.

41. The method of any of claims 1-39, wherein the compound is 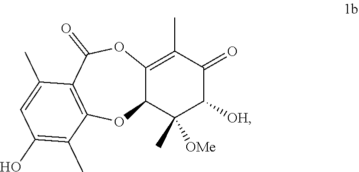
or a pharmaceutically acceptable salt thereof.

42. The method any of claims 1-41, wherein the method further comprises an additional therapeutic agent.

43. The method of claim 42, wherein the additional therapeutic agent is selected from the group consisting of PI3K inhibitor, bromodomain inhibitor, BRAF inhibitor, Parp inhibitor, a SERD, CDK4/CDK6 inhibitor, and Bcl-2 inhibitor.

44. The method of any of claims 1-41, wherein the subject has previously received treatment with a therapeutic agent.

45. The method of claim 44, wherein the therapeutic agent is selected from the group consisting of endocrine therapeutic agent, CDK4/CDK6 inhibitor, an aromatase inhibitor, taxane, and platinum-based therapeutic.

46. The method of any of claims 1-41, wherein the subject is a post-menopausal woman.

47. A method of inhibiting CDK2 comprising contacting a cell with a therapeutically a compound of Formula (I): 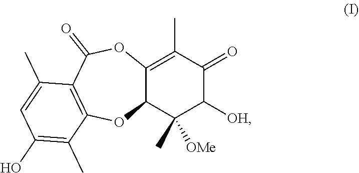
or a pharmaceutically acceptable salt thereof.
